(19) United States
(12) Patent Application Publication
(10) Pub. No.: US 2008/0260716 A1
(43) Pub. Date: Oct. 23, 2008

(54) Method and Composition for High Level Disinfection Employing Quaternary Ammonium Compounds

(75) Inventors: Steven Kritzler, Cronulla (AU); Alex Sava, Paddington (AU)

Correspondence Address: EDWARDS ANGELL PALMER & DODGE LLP, P.O. BOX 55874, BOSTON, MA, 02205, US

(73) Assignee: Nanosonics Pty Limited, Alexandria (AU)

(21) Appl. No.: 11/631,904

(22) PCT Filed: Jul. 5, 2005
(86) PCT No.: PCT/AU05/00997

§ 371 Date: Jan. 28, 2008

(30) Foreign Application Priority Data
Jul. 9, 2004 (AU) 2004903796

(51) Int. Cl. A01N 63/00; A01N 33/12; A01N 33/00; A01N 59/14; A01N 59/24; A01P 1/00; A61L 2/025; A61L 2/08; A61L 2/10; A61L 2/03; A61L 2/18; A01N 43/40; A01N 59/12; A01N 59/00; A01N 37/00

(52) U.S. Cl. 424/94.61; 514/642; 514/635; 514/64; 424/657; 514/588; 514/634; 514/579; 424/722; 424/658; 424/667; 424/718; 514/557; 514/613; 514/277; 424/94.1; 424/94.6; 424/94.63; 422/28; 422/21; 422/24; 422/20; 422/22

(57) ABSTRACT

Methods and compositions for high level disinfection (as herein defined) of a surface. Methods include treating the surface with a composition including a quaternary ammonium compound in a concentration which exceeds 1% w/w and the temperature of treatment is in the range of from 30° C. to 80° C. A log (6) reduction in Mycobacterium terrae is achieved on the surface in less than 10 minutes. The temperature may be produced by a physical chaotrope, a chemical chaotrope (such as) boron or a boron compound or complex or a combination of chaotropic agents. Sequestering agents and enzymes maybe added.

### FIELD OF THE INVENTION

The invention relates to methods and compositions including quaternary ammonium compounds which provide high levels of disinfection

### BACKGROUND OF THE INVENTION

A “high level disinfectant” is a chemical that can be expected to destroy all micro-organisms, with the exception of high numbers of bacterial spores.

Standards have been established for “sterilization”, and for “low”, “intermediate” and “high” level disinfection. These standards are based on the known or possible risk of contamination of a particular medical device by a particular micro-organism, the pathogenic nature of the organism and other principles in infection control. The standards typically require demonstration of sterilization and/or disinfection efficacy against a particular panel of test organisms, which collectively represent the known or possible infection and contamination risks. The test panels and criteria are different for “low”, “intermediate” or “high-level disinfection”. These terms are herein used in accordance with current Food and Drug Administration (“FDA”) criteria for levels of disinfection which are detailed in Premarket Notification [510(k)] Submissions for Liquid Chemical Sterilants and High Level Disinfectants FDA 1997: “A germicide that inactivates all microbial pathogens except large number of bacterial endospores when used according to labelling”. In brief, the FDA regulatory requirement for high level disinfectants includes as the most challenging test a 100% kill of Mycobacterium tuberculosis var. bovis (or a specific strain of a suitable surrogate, such as Mycobacterium terrae) in 400 p.p.m. hard water in the presence of 2% horse serum in a quantitative tuberculocidal test.

Mycobacterium tuberculosis var. bovis is an organism which is refractory to treatment by most bactericidal compounds. In addition, the FDA requirements for high-level disinfectants include efficacy against specific gram-negative and gram-positive bacteria, fungi and viruses. The relevant AOAC sporicidal, tuberculocidal, virucidal and bactericidal tests are referenced in annexure 1 hereto. An additional FDA regulatory requirement for high level disinfectants is that they must also achieve sterilization although a longer exposure time than the disinfection regimen time is permissible. Sterilization is tested with a sporicidal activity test utilizing spores of Bacillus or Clostridium species. It has been demonstrated that the micro-organisms most resistant to chemical sterilants are the spores of Bacillus species B. subtilis. and C. sporogens. Sterilization is a process that completely eliminates or destroys all forms of microbial life, including fungal and bacterial spores. To be used as a high level disinfectant, a chemical must be registered as such with appropriate regulatory authorities such as the FDA (in the USA) or TGA (in Australia).

It is known that a high-level disinfectant (“HLD”) will meet the disinfection efficacy standards of intermediate and low-level disinfection as well. It is universally accepted that low-level disinfection performance cannot predict intermediate or high-level disinfection performance. In fact, it is assumed prior to testing that a low-level disinfectant cannot achieve a higher level disinfection standard.

High level disinfectants are used extensively in the healthcare and medical industry, for example to disinfect endoscopes, kidney dialyzers and other medical instruments and devices, especially those liable to be damaged by heat. They are also extensively used by medical offices and dentists where many of the instruments incorporate rubbers or plastics in their construction and cannot be heated repeatedly to above 60° C. without damage.

Common commercially available high-level disinfectants include glutaraldehyde solutions between 0.3 and 3.4%, which typically require activation with an alkaline buffer just prior to use. Also available are an acidic (pH 1.6-2.0) 7.5%. sup. w/v hydrogen peroxide solution (Sporox®, Reckitt and Colman, Inc.) and an acidic (pH 1.87) mixture of 1.0% hydrogen peroxide plus 0.08% peracetic acid (“PAA”) (Peract™ 20, Minntech Corp. or Cidex OPA®, Johnson & Johnson). The minimum effective concentration of PAA for high-level disinfection at 25 minutes (min) and 20° C. is 0.05% (500 ppm) (Peract™). The minimum effective concentration of peroxide for high-level disinfection at 30 min and 20° C. is 6.0% (Sporox®).

To be acceptable as a high level disinfectant a composition, in addition to meeting regulatory standards of microbiological efficacy, must be compatible with construction materials used in medical instruments such as rubber, plastics, elastomers and metals, and should be easy to use. It is clearly advantageous if the disinfectant has a low order of toxicity and is readily rinseable with water. It should be capable of a simple monitoring and validation procedure. It should have a commercially adequate shelf life and shelf stability. Desirably also it would be economical to manufacture, and achieve high level disinfection in a relatively short time.

No known high level disinfectant meets all of these desiderata. Gluteraldehyde, peracetic acid, and phenols are both obnoxious and toxic. In addition residues of some aldehydes on instruments have been shown to disadvantageously react with biopsy samples and even cause chemically induced anaphylactic shock to patients undergoing endoscopy. Hydrogen peroxide residues have been shown to interfere with cytoscopy samples taken via a disinfected cystoscope, and with biopsy samples taken via an endoscope. Even the most benign high level disinfectants have tended to cause skin irritation or allergenic reaction while others are regarded as potential carcinogens.

Quaternary ammonium compounds (“quats”) have been widely used for industrial and domestic disinfection for many years and are safe and simple to use. Regrettably, although formulations containing quats are known to be effective against gram positive organisms such as streptococcus and staphylococcus, they are among the least effective disinfectants when used alone. Quats are relatively ineffective against gram negative organisms, are notorious for their lack of sporicidal effect, and have been widely reported to have virtually no tuberculocidal activity (see eg “Disinfection, Sterilization, and Preservation”, Seymour S. Block, Fifth edition, page 306). Quats are typically used at concentrations ranging from p.p.m. to 0.25% w/w.

Many workers have screened differently substituted quaternary ammonium compounds, and/or sought coadjuvents, which might raise their effectiveness to a higher disinfection level.

For example U.S. Pat. No. 6,245,361 discloses a combination of 600-800 p. p.m. of a quaternary compound with a chlorine containing compound such as a hypochlorite or diisocyanate in which the chlorine compound provides the tuberculocidal activity. Chlorine compounds are excellent sterilants themselves (at the levels specified in the patent) and it seems that the addition of a quat yields no improvement in Sporicidal/tuberculocidal efficacy when compared to chlorine alone. The improvement claimed is that the combination with the quaternary ammonium compound is said to be “less” toxic and “less” skin irritating than is the chlorine compound alone. However, the presence of the chlorine compound would render the composition corrosive to many construction materials and the combination shares most of the disadvantages of prior art. Disinfectants which contain combinations of active components such as in this example are also disadvantageous with respect to the regulatory process. In some territories although each of the active ingredients may be well known separately with respect to toxicology and materials compatibility, the combination must be treated as a new, previously unknown entity for regulatory purposes.

U.S. Pat. No. 5,444,094 acknowledges that quaternary ammonium salt formulations have long been used as disinfectants but do not display any tuberculocidal activity. Nor do glycol ethers. However, U.S. Pat. No. 5,444,094 teaches that a combination of a quat at 0.1% to 0.2% w/w with at least about 8% w/w/glycol ether is tuberculocidal, while combinations with 6% glycol ether are not. This is surprising, and is attributed to disruption of the mycobacteria trilaminar cell wall which is composed of 60% lipid, by the glycol ether. Glycol ethers are strong solvents and at these high levels are not compatible with vast majority of plastics and rubbers used as materials of construction. Another disadvantage of the U.S. Pat. No. 5,444,094 composition is that the product does not exhibit sporicidal properties (as per AOAC Official Methods of Analysis (1955) sporicidal test, ref No 966.04) and therefore is not a high level disinfectant (“HLD”).

It has been suggested to use disinfectants with ultrasound to kill vegetative spores. Benzethonium chloride at a concentration of 0.25% and at temperatures above 60° C. has been proposed for that use. However as shown herein such treatment is not effective against Mycobacterium and the treatment is not suitable for high level disinfection.

It is current medical device user practice for semi critical medical devices (i.e., those that contact intact skin and mucous membranes such as endoscopes, dental instruments and the like) to use separate short cleaning and disinfecting steps and times, and reusable solutions. Longer soak cleaning or disinfecting times and single-use solutions would for the most part be uneconomical and impractical in current medical or dental practice.

Any discussion of the prior art throughout the specification should in no way be considered as an admission that such prior art is widely known or forms part of common general knowledge in the field.

It is an object of the present invention to provide a high level disinfectant which avoids or ameliorates at least some disadvantages of the prior art. It is an object of preferred embodiments of the invention to provide a high level disinfectant which is shelf stable, effective in a short time, and which poses a significantly reduced occupational health threat.

Preferred embodiments of the invention result in a regulated log 6 reduction in population of Mycobacterium terrae within a time period of 2-10 min in AOAC tuberculocidal tests and both B. subtilis and Clostridium sporogenes spores in under 5 hours as per FDA requirements for high-level disinfection (as defined in Premarket Notification [510(k)] Submissions for Liquid Chemical Sterilants and High Level Disinfectants, FDA 1993).

### BRIEF STATEMENT OF THE INVENTION

According to a first aspect the invention provides a method of high level disinfection (as herein defined) of a surface including the step of treating the surface with a composition including a quaternary ammonium compound and wherein the concentration of said quaternary ammonium compound is selected to exceed 1.0% w/w and the temperature of treatment is selected to be in the range of from 30° C. to 80° C., whereby to achieve log 6 reduction in mycobacterium tuberculosis, if any on the surface, in less than 10 minutes.

In preferred embodiments of the invention, the concentration of quaternary ammonium compound (“quat”) exceeds 2% w/w and preferably greater than 4% w/w. The temperature must be raised to greater than 30° C., preferably to greater than 40° C., and more preferably greater than about 50° C. However the temperature desirably does not exceed about 60° C. in view of the risk of damage to instruments, although with heat resistant materials may be up to 80° C. Preferred selected concentrations and temperatures achieve a log 6 reduction in Mycobacterium terrae in less than 5 minutes.

Those skilled in the art will recognize that quats have hitherto been reported to be incapable of providing high level disinfection. Block (cited supra), a recognized manual in the art of disinfection, says of quats “they are not tuberculocidal or sporocidal or virucidal against hydrophilic viruses at high concentration levels”. When used as a low level “germicide” quats are typically applied to surfaces at ambient temperatures and at concentrations of from about 0.1% up to about 0.25%. There is no suggestion in the prior literature that a quat is capable of killing mycobacterium tuberculosis at any concentration at room temperature, or that increasing temperatures above 30° C. would have any beneficial effect on a quat's biocidal effectiveness.

Indeed the present inventors have found that at ambient temperature and at concentrations of up to 1% w/w quats are not capable of high level disinfection, and even at below 2% w/w they do not achieve that level in a practically short time span. It was therefore surprising to discover that a high level disinfection could be achieved in a practically short time by utilizing a quat and by selecting an appropriate concentration and treatment conditions.

The selected temperature of from 30° C. to 80° C. may be produced by heat or by another physical chaotrope. For example the increase in temperature may be the result of application of heat (which is a chaotrope), or application of a physical chaotropic agent such as electromagnetic radiations (for example ultrasound, microwave, UV, IR or other radiations), electric or magnetic fields, or even shaking or stirring. Other methods of applying energy include electromagnetic radiation or energetic vibration from mechanical means such as magnetic or vortex stirring. Energy may be input from electron beam irradiation, laser, electrolysis, or high energy jets. Selecting a combination of such chaotropic influences may advantageously be utilized. The temperature increase may also be produced by other means, for example, exothermic chemical reaction.

According to a second aspect the invention provides a method according to the first aspect wherein the composition further includes a chemical chaotrope. A preferred chaotrope is boron or a boron compound or complex. Desirably the composition also includes a sequestering agent such as, for example, EDTA.

A chaotropic agent is a physical or chemical interaction with the mixture of quat and microorganisms that tends to increase the solubility of hydrophobic particles in aqueous solutions, or which tends to destabilize aggregations of nonpolar solute particles and micelles, or denatures (folds or unfolds) proteins. Physical chaotropes for use in the invention have been discussed above. Certain chemical chaotropes, such as metal ions, organic and inorganic anions, urea, etc may be used alone with the quat or in combination with a physical chaotrope. For preference a combination of chaotropic agents are employed.

According to a third aspect the invention provides a method according to the first or second aspect wherein the composition further includes an enzyme.

According to a fourth aspect the invention includes a composition for use in a method of high level disinfection (as herein defined) according to any one of the preceding aspects, and including in excess of 1% of a quaternary ammonium compound at 30° C. at a working concentration.

It will be understood that the requirements for achieving high level disinfection imply that the method must be able to meet other requirements defined by the FDA in addition to achievement of log 6 reduction in Mycobacterium tuberculosis var. bovis. Preferred methods according to the invention can also achieve a log 6 reduction in both B. subtilis and Clostridium Sporogenes spores in less than 5 hours in accordance with the appropriate FDA test methods (which specifies less than 24 hours).

According to a fifth aspect the invention provides a high level disinfectant comprising a quaternary ammonium compound in a concentration greater than 1% at a working strength and in combination with one or more chemicals which is a chaotropic agent but is not a spore opening chemical.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### PREFERRED EMBODIMENTS OF THE INVENTION

The invention will now be more particularly described by way of example only.

The invention employs a quaternary ammonium compound under selected conditions to achieve high level disinfection. Any commercially available quaternary compounds are suitable in the present invention.

A quaternary ammonium compound can be represented by the general formula (R₁R₂R₃R₄N+)X⁻. R₁, R₂, R₃ and R₄ can independently be any suitable substituted or unsubstituted linear or cyclic groups such as alkyl, aryl, alkaryl, aralkyl, ether and the like.

Preferably, in the present invention R₁ and R₂ and are independently chosen from the group consisting of alkyl groups having 1 to 3 carbon atoms, R₃ is chosen from the group consisting of alkyl groups having 8 to 20 carbon atoms, and R₄ is chosen from the group consisting of alkyl groups having 8 to 20 carbon atoms, aryl groups and aryl-substituted alkyl groups where said substituted alkyl groups have 1-3 carbon atoms and X⁻ is chosen so as to render said quaternary ammonium compound water-soluble. Any suitable quaternary compound may be used but, for preference, the quaternary compound used in the invention is a dialkyl quaternary compound and more preferably is a quaternary compound in which one of the alkyls has a chain length of less than 18. Desirably at least one of the alkyls is a C₁₄-C₁₈ alkyl with C₁₂ highly preferred. The quat may have more than one alkyl chain, or may be an aryl quat. The quat may be, for example, CHG.

Counterion X⁻ may be any suitable counterion, inorganic or organic. Suitable examples of X⁻ may include, but are not limited to halide (fluoride, chloride, bromide or iodide), hydroxide, tetrafluoroborate, phosphate, or carbonate.

The term quat as used herein also encompasses mixtures of quaternary ammonium compounds.

In preferred forms of the invention the selected conditions include application of a combination of chaotropic agents. For example, a quat at 4% w/w is used in combination with a boron compound together with heat or heat and ultrasound, at say 50° C. Or to use another example, a quat at 5% w/w may be used with a surfactant and/or a suitable solvent, together with an input of energy such as to increase temperature to 40° C. It is unclear whether the input of energy, such as from heating, assists in driving the folding/unfolding equilibrium in favour of unfolding the spore coat and Mycobacterium cell membrane proteins/lipoproteins, or whether it merely assists in providing momentary access of quat molecules to otherwise “inaccessible” parts of the spore surface, or overcomes inherent to quats inactivation by proteinaceous matter or whether it is effective in activating the quat or targeted microorganisms in some other manner. In highly preferred forms of the invention a quat is used together with a protease in the presence of borax and at elevated temperature.

The selected conditions include energy input to increase the temperature from 30° C. to 80° C., preferably above 40° C. and below 60° C. Temperatures above 60° C. are not desirable because of the detrimental effect of temperature on construction materials of thermosensitive medical instruments. The temperature may be elevated by application of heat, but energy input may be by means of application of ultrasonic energy, infrared or microwave radiation, high pressure, the action of electric and/or magnetic fields, and even shaking or stirring all of which may be influential in promoting unfolding (refolding).

Chemical chaotropes which may be combined with the quat include:

(1) Selected organic solvents of a kind which tend to denature, dissolve or swell proteins. Generally the products are not completely unfolded and possess an ordered conformation which differs from the native state. Solvents which favour helical conformations (i.e. unfolding) are exemplified by N-dimethylformamide, formamide, m-cresol, dioxan, CHCl₃, pyridine, dichlorethylene, and 2-chloroethanol. This group also includes solvents which have a weak tendency to form hydrogen bonds such as the alcohols, ethanol, n-propanol, methanol (especially in mixture with 0.01% HCl), Also, solvents which tend to disorganize the structure e.g. dimethylsulfoxide (DMSO) at high concentrations, dichloroacetic acid and trifluoroacetic acid, and other electrophilic solvents. It should be noted that the vast majority of these compounds actually strengthen the spore coat as opposed to acting as a spore-opener.

(2) Certain organic solutes and chaotropic agents, such as urea or guanidine hydrochloride (GuHCl). The transition to randomly coiled polypeptide is complete for 6-8M GuHCl at room temp except for some exceptionally stable proteins. These agents may be markedly influenced by temperature, pH other reagents and conditions.

Inorganic salts can induce conformational transitions in proteins. For example LiBr, CaCl₂, KSCN, NaI, NaBr, borax, sodium azide are strong denaturants. Although these salts do not necessarily lead to completely unfolded protein, the residual ordered structure may be disrupted by energy input e.g. increasing temperature. Anions such as CNS>I⁻>Br⁻>NO₃⁻>Cl⁻>CH₃COO⁻>SO₄²⁻ exhibit similar behaviour as do guanidinium salts and tetraalkyl ammonium salts However (GuH)₂SO₄ has been observed to protect certain proteins against denaturation. Boron may be used in the form of a compound or complex.

Adsorption on certain surfaces and interfaces including zeolites, including air/liquid interfaces.

(3) Enzymes, for example proteases, amylase, lipidases, cellulases and the like.

### EXAMPLES OF THE INVENTION

In the following tables unless otherwise specified references to “kill time” mean the time required to achieve complete kill as defined in the relevant AOAC test (more particularly identified in annexure 1). “No kill” means less than 2 log reduction from the initial population. Unless otherwise specified “QUAT” is benzalkonium chloride, specifically Gardiquat NC-50 (Albright & Wilson). Test points for vegetative bacteria (M. terrae) were 2, 5, 10, 20 and 60 mins. Test points for spores were 0.5, 1, 2, 4, 16, 24, 48 hours. The last column in each table indicates whether the tested combination satisfies the FDA requirement for high level disinfection (“HLD”) or fails to do so (“F”).

TABLE 1
Examples of prior art

| | | | Kill Time | | | |
|---|---|---|---|---|---|---|
| Test | | | Mycobacterium | Kill time | Kill Time | HLD |
| number | Formulation | Conditions | terrae | B. subtilis | C. sporogens | or F |
| 1.1 | 0.025% QUAT | 25° C. | >60 mins | >24 hrs | >24 hrs | F |
| 1.2 | 0.25% QUAT | 25° C. | >60 mins | >24 hrs | >24 hrs | F |
| 1.3 | 0.25% | 62° C. + | >60 mins | >24 hrs | nt | F |
| | benzethonium | ultrasound | | | | |
| | chloride, | 20 min/hr | | | | |
| | 0.25% Triton | | | | | |
| | X-100, 5% | | | | | |
| | isopropanol | | | | | |

Table 1 shows examples of the use of quats according to prior art in which concentrations in the range Of 0.025% to 0.25% are employed at ambient temperature. It can be seen that at up to 0.25% (which is considered a high concentration for formulation of quats) the “kill time” of Mycobacterium is greater than an hour at 25° C., and for B. subtilis and C. sporogenes kill time is greater than 24 hrs. As test 1.3 shows, the result is the same at 62° C., even in the presence of ultrasound. None of the examples in Table 1 could be considered useful for High Level Disinfection.

Table 2 shows some examples according to the invention.

TABLE 2
Examples according to invention

| | | | Kill Time | | | |
|---|---|---|---|---|---|---|
| Test | | | Mycobacterium | Kill Time | Kill Time | HLD |
| number | Formulation | Conditions | terrae | B. subtilis | C. sporogens | or F |
| 2.1 | 5% QUAT | 30° C. | <5 mins | <2 hrs | <2 hrs | HLD |
| 2.2 | 1.0% QUAT | 40° C. | <5 mins | <2 hrs | <2 hrs | HLD |
| 2.3 | 2.0% QUAT | 40° C. | <5 mins | <2 hrs | <2 hrs | HLD |
| 2.4 | 5.0% QUAT | 40° C. | <5 mins | <1 hrs | <1 hrs | HLD |
| 2.5 | 1.0% QUAT | 50° C. | <5 mins | <1 hrs | <2 hrs | HLD |
| 2.6 | 2.0% QUAT | 50° C. | <5 mins | <1 hrs | <2 hrs | HLD |
| 2.7 | 5.0% QUAT | 50° C. | <5 mins | <1 hrs | <1 hrs | HLD |
| 2.8 | 1.0% QUAT | 80° C. | <5 mins | <1 hrs | <1 hrs | HLD |

Astonishingly, in contrast to the examples of Table 1, at above 1% w/w of quat and at 40° C., a kill time of Mycobacterium terrae of less than 5 mins can be achieved, and of B. subtilis and C. sporogenes of less than 2 hours, reducing to less than one hour at 5% and 50° C. or 1% at 80° C. All the table 2 examples give high level disinfection.

The present inventor showed that increasing the temperature from 25° C. up to 60° C. had no beneficial effect on a Quats ability to kill Mycobacterium terrae at the prior art concentrations of 0.25% w/w. (table 3 tests 3.1-3.3), with or without ultrasound,

TABLE 3
Examples outside the invention

| Test | | | Kill Time | Kill Time | Kill Time | HLD |
|---|---|---|---|---|---|---|
| number | Formulation | Conditions | M. terrae | B. subtilis | C. sporogens | Or F |
| Prior art | 0.25% QUAT | 25° C. | >60 mins | >24 hrs | >24 hrs | F |
| 3.1 | 0.25% QUAT | 40° C. | >60 mins | >24 hrs | >24 hrs | F |
| 3.2 | 0.25% QUAT | 50° C. | >60 mins | >24 hrs | >24 hrs | F |
| 3.3 | 0.25% QUAT | 60° C. | >60 mins | >24 hrs | >24 hrs | F |
| 3.4 | 0.25% QUAT | 62° C. + | >60 mins | >24 hrs | >24 hrs | F |
| | | ultrasound | | | | |
| 3.5 | 1.0% QUAT | 25° C. | >60 mins | >48 hrs | >48 hrs | F |
| | | | | (no kill) | (no kill) | |
| 3.6 | 2.0% QUAT | 25° C. | >60 mins | >48 hrs | >48 hrs | F |
| | | | | (no kill) | (no kill) | |
| 3.7 | 5.0% QUAT | 25° C. | >60 mins | >48 hrs | >48 hrs | F |
| | | | | (no kill) | (no kill) | |
| 3.8 | 0.6% | 40° C. | >60 mins | >24 hrs | >24 hrs | F |
| 3.9 | 0.6% QUAT | 50° C. | >20 mins | <16 hrs | <16 hrs | F |

Likewise, tests 3.5-3.9 show that increasing concentration from 0.25% (1 in 400) to 5.0% (1 in 20) which is about an twenty fold increase above the concentrations used in the prior art had no significant effect at 25° C.

The present inventors were thus surprised to discover that at about 50° C. and at a concentration of above 0.6%, the kill time of Mycobacterium terrae fell suddenly and sharply to between 20 and 60 minutes, and of B. subtilis and C. sporogenes to less than 16 hours. These times are not sufficient for practical high level disinfection. For practical high level disinfection a combination of a concentration greater than 1% and an increase in temperature above room temperature, and preferably above 30° C., more preferably above 40° C. (or equivalent chaotropic effect) must be selected.

Table 4 exemplifies the effect of a chemical chaotrope, in this case boron.

TABLE 4
Effect of Borax

| Test | | | Kill Time | Kill Time | Kill Time | HLD |
|---|---|---|---|---|---|---|
| number | Formulation | Conditions | M. terrae | B. subtilis | C. sporogens | or F |
| 4.1 | 2% QUAT | 25° C. + 1% | >60 min | >48 hrs | >48 hrs | F |
| | | borax | | (no kill) | (no kill) | |
| 4.2 | 5% QUAT | 25° C. + 1% | >60 min | >48 hrs | >48 hrs | F |
| | | borax | | (no kill) | (no kill) | |
| 4.3 | 1% QUAT | 40° C. + 1% | <5 min | <1 hr | <1 hr | HLD |
| | | borax | | | | |
| 4.4 | 2% QUAT | 40° C. + 1% | <2 min | <1 hr | <1 hr | HLD |
| | | borax | | | | |
| 4.5 | 5% QUAT | 40° C. + 1% | <2 min | <1 hr | <1 hr | HLD |
| | | borax | | | | |
| 4.6 | 2% QUAT | 50° C. + 1% | <2 min | <1 hr | <1 hr | HLD |
| | | borax | | | | |
| 4.7 | 5% QUAT | 50° C. + 1% | <2 min | <1 hr | <1 hr | HLD |
| | | borax | | | | |

Tests 4.1 and 4.2 are at 25° C. and therefore outside the selected range of the invention. However the results for tests 4.3-4.7 selected according to the invention are in stark contrast to tests 4.1 and 4.2.

Table 5 shows the effect of ultrasound.

TABLE 5
Effect of ultrasound

| Test | | | Kill Time | Kill Time | Kill Time | HLD |
|---|---|---|---|---|---|---|
| number | Formulation | Conditions | M. terrae | B. subtilis | C. sporogens | Or F |
| 5.1 | 1% QUAT | 40° C. + | <5 min | <2 hrs | <2 hrs | HLDs |
| | | ultrasound 10 | | | | |
| | | min each hour | | | | |
| 5.2 | 2% QUAT | 40° C. + | <5 min | <1 hr | <1 hr | HLD |
| | | ultrasound 10 | | | | |
| | | min each hour | | | | |
| 5.3 | 5% QUAT | 40° C.+ | <5 min | <1 hr | <1 hr | HLD |
| | | ultrasound 10 | | | | |
| | | min each hour | | | | |
| 5.4 | 1% QUAT | 40° C. + | <5 min | <1 hr | <1 hr | HLD |
| | | ultrasound 10 | | | | |
| | | min each hour | | | | |
| 5.5 | 2% QUAT + | 40° C. + | <2 min | <1 hr | <1 hr | HLD |
| | 1% borax | ultrasound 10 | | | | |
| | | min each hour | | | | |
| 5.6 | 5% QUAT + | 40° C.+ | <2 min | <1 hr | <1 hr | HLD |
| | 1% borax | ultrasound 10 | | | | |
| | | min each hour | | | | |
| 5.7 | 2% QUAT + | 40° C. + | <2 min | <1 hr | <1 hr | HLD |
| | 1% borax + | ultrasound 10 | | | | |
| | Protease | min each hour | | | | |

Comparison of tests 5.2 with tests 2.2 & 2.3 shows the beneficial effect of ultrasound in combination with heat, while tests 5.5-5.7 show the combined effect of chemical and physical chaotropes. The combination of examples 5.5-5.7 reduce kill time to less than two minutes for M. terrae. Experiment 5.7 shows that the result is obtainable in the presence of protease.

Column 2 of table 6 shows a concentration of Quat combined with 0.2% of protease, while column 3 shows the temperature, borax concentration (if any), and presence or absence of ultrasound. Again it can be seen that at 25° C. neither protease, borax, nor ultrasound have a significant benefit even at up to 2% quat concentration, but that at higher temperatures a surprising and dramatic change in kill time occurs at 2%.

TABLE 6
Effect of protease, borax, ultrasound

| | 0.2% Protease | | | | | |
|---|---|---|---|---|---|---|
| | (Subtisilin)* | | | | | |
| | plus QUAT | | | | | |
| Test | conc. as | | Kill Time | Kill Time | Kill Time | HLD |
| number | shown | Conditions | M. terrae | B. subtilis | C. sporogens | Or F |
| 6.1 | 2% | 25° C. | >60 min | >48 hrs | >48 hrs | F |
| | | | | (no kill) | (no kill) | |
| | 2% | 25° C. + 1% | >60 min | >48 hrs | >48 hrs | F |
| | | borax | | (no kill) | (no kill) | |
| 6.2 | 2% | 40° C. | <5 min | <2 hr | <1 hr | HLD |
| 6.3 | 2% | 40° C. + 1% | <2 min | <1 hr | <1 hr | HLD |
| | | borax | | | | |
| 6.4 | 2% | 50° C. | <5 min | <1 hr | <1 hr | HLD |
| 6.5 | 2% | 50° C. + 1% | <2 min | <1 hr | <1 hr | HLD |
| | | borax | | | | |
| 6.6 | 2% | 40° C.+ | <2 min | <1 hr | <1 hr | HLD |
| | | ultrasound | | | | |
| | | 10 min | | | | |
| | | each hour + | | | | |
| | | 1% borax | | | | |
| 6.7 | 2% QUAT + | 40° C. + | <2 min | <1 hr | <1 hr | HLD |
| | 1% borax + | ultrasound | | | | |
| | Protease | 10 min | | | | |
| | | each hour | | | | |

*Savinase 16 L

Table 7 shows that similar results are obtained with other quats:—

TABLE 7
Other Quats

| Test | | | Kill Time | Kill Time | Kill Time | HLD |
|---|---|---|---|---|---|---|
| number | Formulation | Conditions | M. terrae | B. subtilis | C. sporogens | or F |
| 7.1 | 1% Quat(1) | 50° C. | <10 min | <2 hrs | <2 hrs | HLD |
| 7.2 | 1% Quat(2) | 50° C. | <10 min | <2 hr | <2 hr | HLD |
| 7.3 | 1% Quat(3) | 50° C. | <10 min | <2 hr | <2 hr | HLD |
| 7.4 | 1% Quat(4) | 50° C. | <10 min | <2 hr | <2 hr | HLD |

(Quat 1) is Didecyl Dimethyl Ammonium Chloride twin chain (Bardac 2280 from Lonza),
(Quat 2) is Dioctyl Dimethyl Ammonium Chloride-twin chain quat (Bardac LF-80 from Lonza),

### (Quat 3) is Barquat MB-50 (N-Alkyl Dimethyl Ammonium Chloride, C14—50%, C12—40%, C16—10%), and

(Quat 4) is Dodigen 228 LF (N-Alkyl Dimethyl Ammonium Chloride, C14—60%, C12—30%, C10—10%) single chain quat.

As will be apparent to those skilled in the art from the teaching hereof, quats other than those exemplified may be used, or quats may be combined for the purposes of the invention. In preferred embodiments the quat will be formulated with one or more chaotropes for example boron or a boron compound, enzymes and or surfactants and within the selected range of concentrations or may be formulated as a concentrate intended to be diluted so as to have a concentration of the magnitude selected at the working dilution. While increasing the temperature has a straight forward chaotropic effect, use of microwave, ultrasonic, infrared or other electromagnetic radiation alone or in combination with chemical chaotropic agents may be used.

### Annexure 1

AOAC tests for determination of High level Disinfection as defined in current FDA criteria (detailed in “Premarket Notification [510(k)] Submissions for Liquid Chemical Sterilants and High Level Disinfectants FDA 1993”.):

AOAC sporicidal test: AOAC Ref No 966.04, AOAC Official Methods of Analysis.

### AOAC Tuberculocidal Activity of Disinfectants

### AOAC Ref No 965.12, AOAC Official Methods of Analysis (1995)

### AOAC Hard Surface Carrier Test₁₀

### AOAC Ref Nos 991.47, 991.48 and 991.49, AOAC Official Methods of Analysis (1995).

### AOAC Germicidal Spray Products Test₁₁

### AOAC Ref No 961.02, AOAC Official Methods of Analysis (1995)

### AOAC Fungicidal Test

AOAC Ref No 955.17, AOAC Official Methods of Analysis (1995)

1. A method of high level disinfection of a surface including the step of treating the surface with a composition including a quaternary ammonium compound wherein the concentration of said quaternary ammonium compound exceeds 1% w/w and the temperature of treatment is in the range of from 30° C. to 80° C., whereby to achieve log 6 reduction in Mycobacterium terrae or Mycobacterium tuberculosis, if any, on the surface in less than 10 minutes.

2. A method according to claim 1 wherein the composition achieves log 6 reduction in Mycobacterium terrae or Mycobacterium tuberculosis, if any, on the surface in less than 5 minutes.

3. A method according to claim 1 wherein the concentration of quaternary ammonium compound exceeds 2% w/w.

4. A method according to claim 3 wherein the concentration of quaternary ammonium compound exceeds 4% w/w.

5. A method according to claim 1 wherein the quaternary ammonium compound is (R₁R₂R₃R₄N⁺)X⁻ where R₁, R₂, R₃ and R₄ are independently substituted or unsubstituted linear or cyclic groups.

6. A method according to claim 5 wherein R₁, R₂, R₃ and R₄ are independently alkyl, aryl, alkaryl, aralkyl or ether.

7. A method according to claim 6 wherein R₁ and R₂ and are independently chosen from the group consisting of alkyl groups having 1 to 3 carbon atoms, R₃ is chosen from the group consisting of alkyl groups having 8 to 20 carbon atoms, and R₄ is chosen from the group consisting of alkyl groups having 8 to 20 carbon atoms, aryl groups and aryl-substituted alkyl groups wherein said substituted alkyl groups have 1-3 carbon atoms and X⁻ is chosen so as to render said quaternary ammonium compound water-soluble.

8. A method according to claim 6 wherein at least one of R₁, R₂, R₃ and R₄ is a C₁₄-C₁₈ alkyl group.

9. A method according to claim 6 wherein at least one of R₁, R₂, R₃ and R₄ is a C₁₂ alkyl group.

10. A method according to claim 1 wherein the quaternary ammonium compound is chlorhexidine gluconate.

11. A method according to claim 5 wherein X⁻ is an inorganic or organic counterion.

12. A method according to claim 11 wherein X⁻ is halide, hydroxide, tetrafluoroborate, phosphate, or carbonate.

13. A method according to claim 1 wherein the quaternary ammonium compound is a mixture of quaternary ammonium compounds.

14. A method according to claim 1 wherein the temperature is greater than 40° C.

15. A method according to claim 1 wherein the temperature is greater than 50° C.

16. A method according to claim 1 wherein the temperature does not exceed 60° C.

17. A method according to claim 1 wherein the temperature of from 30° C. to 80° C. is produced by a physical chaotrope.

18. A method according to claim 17 wherein the physical chaotrope is heat.

19. A method according to claim 18 wherein the heat is produced by an exothermic chemical reaction.

20. A method according to claim 17 wherein the chaotropic agent is electromagnetic radiation, ultrasound, shaking or stirring.

21. A method according to claim 20 wherein the chaotropic agent is microwave radiation, UV radiation, IR radiation, an electric field, a magnetic field, electron beam irradiation, laser, electrolysis, magnetic stirring, vortex stirring, high energy jets, adsorption on active surfaces and interfaces.

22. A method according to claim 1 wherein the temperature of from 30° C. to 80° C. may is produced by a chemical chaotrope.

23. A method according to claim 22 wherein the chemical chaotrope is a metal or metal ion, an organic ion or an inorganic ion.

24. A method according to claim 22 wherein the chemical chaotrope is boron or a boron compound or complex.

25. A method according to claim 1 wherein a combination of chaotropic agents are employed.

26. A method according to claim 25 wherein a combination of physical and chemical chaotropic agents are used.

27. A method according to claim 1 wherein the composition further includes a sequestering agent.

28. A method according to claim 27 wherein the sequestering agent is EDTA.

29. A method according to claim 1 wherein the composition further includes an enzyme.

30. A composition according to claim 1 when used in a method of high level disinfection (as herein defined) according to any one of the proceeding claims including in excess of 1% of a quaternary ammonium compound at 30° C. at a working concentration.

31. A composition according to claim 30 further including a substance which is a chaotropic agent but is not a spore opening chemical.

32. A composition according to claim 30 wherein the concentration of quaternary ammonium compound exceeds 2% w/w.

33. A composition according to claim 30 wherein the concentration of quaternary ammonium compound exceeds 4% w/w.

34. A composition according to claim 30 wherein the quaternary ammonium compound is (R₁R₂R₃R₄N⁺)X⁻ where R₁, R₂, R₃ and R₄ are independently any suitable substituted or unsubstituted linear or cyclic group.

35. A composition according to claim 34 wherein R₁, R₂, R₃ and R₄ are independently alkyl, aryl, alkaryl, aralkyl or ether.

36. A composition according to claim 34 wherein R₁ and R₂ and are independently chosen from the group consisting of alkyl groups having 1 to 3 carbon atoms, R₃ is chosen from the group consisting of alkyl groups having 8 to 20 carbon atoms, and R₄ is chosen from the group consisting of alkyl groups having 8 to 20 carbon atoms, aryl groups and aryl-substituted alkyl groups where said substituted alkyl groups have 1-3 carbon atoms and X⁻ is chosen so as to render said quaternary ammonium compound water-soluble.

37. A composition according to claim 34 wherein one of R₁, R₂, R₃ and R₄ is an alkyl group with a chain length of less than 18.

38. A composition according to claim 34 wherein one of R₁, R₂, R₃ and R₄ is a C₁₄-C₁₈ alkyl group.

39. A composition according to claim 34 wherein one of R₁, R₂, R₃ and R₄ is a C₁₂ alkyl group.

40. A composition according to claim 30 wherein the quaternary compound is a dialkyl quaternary compound.

41. A composition according to claim 34 wherein X⁻ is an inorganic or organic counterion.

42. A composition according to claim 41 wherein X⁻ is halide, hydroxide, tetrafluoroborate, phosphate, or carbonate.

43. A composition according to claim 30 wherein the quaternary compound is chlorhexidine gluconate.

44. A composition according to claim 30 including a mixture of quaternary ammonium compounds.

45. A composition according to claim 30 including one or more components for an exothermic chemical reaction.

46. A composition according to claim 30 further including a chemical chaotrope.

47. A composition according to claim 46 wherein the chemical chaotrope is a metal ion, organic or inorganic anions.

48. A composition according to claim 47 wherein the chemical chaotrope is boron or a boron compound or complex

49. A composition according to claim 47 wherein the chemical chaotrope is urea, a guanidinium salt or a tetraalkyl ammonium salt.

50. A composition according to claim 47 wherein the chemical chaotrope is guanidine hydrochloride.

51. A composition according to claim 47 wherein the chemical chaotrope is LiBr, CaCl₂, KSCN, NaI, NaBr, borax, sodium azide

52. A composition according to claim 47 wherein the chemical chaotrope is an anion selected from CNS⁻, I⁻, Br⁻, NO₃⁻, Cl⁻, CH₃COO⁻ or SO₄²

53. A composition according to claim 46 wherein the chemical chaotrope is an organic solvent of a kind which tend to denature, dissolve or swell proteins.

54. A composition according to claim 53 wherein the chemical chaotrope is N-dimethylformamide, formamide, m-cresol, dioxan, CHCl₃, pyridine, dichlorethylene, or 2-chloroethanol.

55. A composition according to claim 46 wherein the chemical chaotrope is a solvent which has a weak tendency to form hydrogen bonds.

56. A composition according to claim 55 wherein the chemical chaotrope is an alcohol.

57. A composition according to claim 56 wherein the chemical chaotrope is ethanol, n-propanol, methanol, ethanol/0.01% HCl, n-propanol/0.01% HCl or methanol/0.01% HCl.

58. A composition according to claim 46 wherein the chemical chaotrope is a structurally disorganising solvent.

59. A composition according to claim 58 wherein the chemical chaotrope is dimethylsulfoxide (DMSO), dichloroacetic acid, trifluoroacetic acid or an electrophilic solvent.

60. A composition according to claim 46 wherein a combination of chaotropic agents are employed.

61. A composition according to claim 30 further including a complexing agent.

62. A composition according to claim 61 wherein the complexing agent is EDTA.

63. A composition according to claim 30 further including an enzyme.

64. A composition according to claim 63 wherein the enzyme is a protease, amylase, lipidases or cellulase.

65. A composition according to claim 30 including a protease and borax.
